(19) United States
(12) Patent Application Publication
(10) Pub. No.: US 2013/0143800 A1
(43) Pub. Date: Jun. 6, 2013

(54) COMBINATION THERAPIES TO TREAT DIABETES

(71) Applicant: Research Development Foundation, Carson City, NV (US)

(72) Inventors: Marc MONTMINY, San Diego, CA (US); Sam VAN DE VELDE, La Jolla, CA (US)

(73) Assignee: RESEARCH DEVELOPMENT FOUNDATION, Carson City, NV (US)

(21) Appl. No.: 13/670,980

(22) Filed: Nov. 7, 2012

Related U.S. Application Data
(60) Provisional application No. 61/556,656, filed on Nov. 7, 2011.

(51) Int. Cl. A61K 38/22; A61K 45/00; A61K 38/28

(52) U.S. Cl. CPC A61K 38/22; A61K 38/28; A61K 45/00
USPC 514/5.9; 435/375; 514/7.2

(57) ABSTRACT

Provided are methods for treating diabetes comprising administering to a patient a GLP-1 agonist and an iron chelator. In various embodiments, methods are provided for culturing pancreatic beta islet cells comprising contacting the beta cells with a GLP-1 agonist and an iron chelator in an amount effective to promote survival of the beta cells.

This application claims the benefit of U.S. Provisional Patent Application No. 61/556,656, filed Nov. 7, 2011, the entirety of which is incorporated herein by reference.

This invention was made with government support under R01-DK049777 and R01-DK083834 awarded by the National Institutes of Health. The government has certain rights in the invention.

### BACKGROUND OF THE INVENTION

1. Field of the Invention

The present invention relates generally to the fields of molecular biology and medicine. More particularly, it concerns methods of treating diabetes with a GLP-1 agonist and an iron chelator.

2. Description of Related Art

Diabetes is an epidemic that continues to affect millions of people worldwide. In 2000, according to the World Health Organization, at least 171 million people worldwide suffer from diabetes, or 2.8% of the population. The incidence of diabetes is increasing, and it is estimated that by 2030, this number may almost double. In the U.S. alone, more than 26 million people have diabetes, and approximately 90-95% of these people have type II diabetes.

Significant efforts have been made to develop compounds to treat type II diabetes. One class of compounds that have been recently developed is agonists of the incretin hormone glucagon-like peptide-1 (GLP-1). These agents exploit the physiological effects of GLP-1 and can in some patients alleviate various pathophysiological features of type 2 diabetes, such as enhancing glucose-dependent insulin secretion by pancreatic beta-cells, suppressing inappropriately elevated glucagon secretion, and slowing gastric emptying, although the exact mechanism of these effects is not fully understood. Some adverse effects with GLP-1 agonists have been observed, including adverse gastrointestinal effects such as sour stomach, belching, diarrhoea, heartburn, indigestion, nausea, vomiting, as well as dizziness, feeling jittery, and headache. Additionally, GLP-1 agonists are not effective in all patients with type II diabetes. Clearly, there is a need for improved methods for treating type II diabetes.

### SUMMARY OF THE INVENTION

The present invention overcomes limitations in the prior art by providing new methods for treating diabetes and promoting beta cell survival. The invention is based, in part, on the identification that GLP-1 can promote viability of pancreatic cells by triggering a delayed wave of transcription that proceeds via hypoxia inducible factor-1 (HIF1). More specifically, increases in cAMP promote resulting from GLP-1 activation cause the accumulation of HIF1α in beta cells by activating the mTOR pathway. These findings support the use of a GLP1 agonist (e.g., exenatide) in combination with a compound to promote HIF1α, such as an iron chelator or cAMP, to promote viability of pancreatic islet cells and/or treat diabetes. In various embodiments, the combination of a GLP-1 agonist and an iron chelator may be used to treat a patient with type I or type II diabetes.

An aspect of the present invention relates to a method of treating diabetes in a subject comprising administering to the subject a GLP-1 agonist and an iron chelator in an amount effective to treat diabetes. The GLP-1 agonist may be selected from the group consisting of exenatide, bydureon, liraglutide, albiglutide, taspoglutide, and lixisenatide. In some embodiments, the GLP-1 agonist is exenatide. The iron chelator may be selected from the group consisting of deferoxamine and deferasirox. The subject may be a human, such as a human with type II diabetes. The GLP-1 agonist may be administered subcutaneously to the subject. The method may further comprise administering an additional diabetes therapy to the subject. The additional diabetes therapy may comprise administration of insulin to the subject or administration of a dipeptidyl peptidase-4 inhibitor to the subject. The dipeptidyl peptidase-4 inhibitor may be selected from the group consisting of metformin, sitagliptin (MK-0431), vildagliptin (LAF237), saxagliptin, linagliptin, dutogliptin, gemigliptin, berberine, and alogliptin.

Another aspect of the present invention relates to a method of culturing beta islet cells in vitro, comprising contacting the beta islet cells with a GLP1 agonist and a HIF1 activator in an amount effective to promote survival of the beta cells. The GLP1 agonist may be exenatide. The HIF1 activator may be a cAMP agonist, such as, e.g., forskolin. The cAMP agonist, e.g., forskolin, may be present in the pharmaceutical preparation in an amount sufficient to promote HIF-1α activity or survival of the beta cells. In some embodiments, the HIF1 activator is an iron chelator. In some embodiments, the iron chelator is selected from the group consisting of deferoxamine and deferasirox. Said amount may be effective to promote proliferation of the beta islet cells. The method may further comprise administering a plurality of the cultured beta islet cells to a subject. The cultured beta islet cells may be comprised in a pharmaceutical preparation such as, e.g., a pharmaceutical preparation formulated for intravenous or subcutaneous administration. The subject may be a human, such as a subject or patient with type II diabetes.

Yet another aspect of the present invention relates to a pharmaceutical preparation comprising a GLP-1 agonist and an iron chelator. The pharmaceutical preparation may be formulated for intravenous or subcutaneous administration. In some embodiments, the GLP-1 agonist is selected from the group consisting of exenatide, bydureon, liraglutide, albiglutide, taspoglutide, and lixisenatide. In some embodiments, the GLP-1 agonist is exenatide. The preparation may comprise a cAMP agonist, such as, e.g., forskolin. The iron chelator may be selected from the group consisting of deferoxamine and deferasirox.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F: Serial Induction of CREB and HIF pathways by cAMP in beta cells. FIG. 1A. and FIG. 1B. Profiles of mRNA accumulation for early (FIG. 1A) and late (FIG. 1B) cAMP inducible genes in INS-1 insulinoma cells exposed to FSK for 2 or 16 hours, respectively. Representative genes upregulated in response to FSK indicated below for each group. FIG. 1C. and FIG. 1D. Q-PCR analysis of early (FIG. 1C) and late (FIG. 1D) cAMP responsive genes in primary mouse islets exposed to FSK for 2 or 16 hours. E. and F. Transient assay of CREB (FIG. 1E) and HIF (FIG. 1F) reporter activity in INS-1 cells exposed to FSK for 6 or 20 hours. G. and H. Effect of cycloheximide (CHX) on mRNA amounts for early (NR4A2) and late (HMOX1) cAMP response genes in INS-1 cells. (*; P<0.05). Data are means±s.d.

FIGS. 2A-F: GLP-1 stimulates HIF activity via induction of mTOR. FIG. 2A, Immunoblots showing time course of HIF1α and phospho (Ser133) CREB accumulation in INS-1 cells exposed to FSK. FIG. 2B, Effect of GLP-1 agonist Exendin-4 and 20 mM glucose or 2-deoxy glucose (2dglc), alone and in combination, on HIF1α protein amounts (top) and on HRE-luciferase reporter activity (bottom) in INS-1 cells. FIG. 2C, Chromatin immunoprecipitation (ChIP) assay showing effect of FSK on recruitment of the CREB coactivator CRTC2 (top) or HIF1α (bottom) to early (NR4A2) and late (HMOX1, GLUT1) cAMP responsive genes in INS-1 cells. FIG. 2D, Effect of HIF1α over-expression (top) or RNAi-mediated depletion (bottom) on GLUT1 mRNA amounts in INS-1 cells exposed to FSK for 2 or 16 hours. FIG. 2E, Effect of FSK and DMOG, alone or together, on HIF1α and phospho-S6 protein amounts (top) and on HRE-luc reporter activity (bottom) in INS-1 cells. Exposure to rapamycin indicated. FIG. 2F, Effect of rapamycin on HMOX1 mRNA amounts (top) and on recruitment of HIF1α to the HMOX1 promoter (bottom) in INS-1 cells exposed to FSK.

FIGS. 3A-D: cAMP stimulates mTOR via CREB-dependent increases in IRS2-AKT signaling. FIG. 3A, Top, immunoblot showing IRS2 protein amounts in INS-1 cells exposed to FSK. Bottom, immunoblot showing effect of dominant negative A-CREB expression on IRS2-AKT signaling and HIF1α accumulation in INS-1 cells. FIG. 3B. Effect of A-CREB expression on HMOX1 mRNA levels in INS-1 cells exposed to FSK. FIG. 3C, Immunoblot showing effects of FSK on AKT activation and TSC2 phosphorylation in INS-1 cells exposed to FSK for 16 hours. Effects of PI3-kinase inhibitor LY294002 (LY) indicated. FIG. 3D, Immunoblots showing effects of IRS1 and IRS2 over-expression (left) or RNAi-mediated knockdown (right) on AKT activation and HIF1α accumulation in INS-1 cells. Exposure to FSK indicated.

FIGS. 4A-F: The mTOR-HIF pathway mediates effects of GLP-1 on islet cell viability. FIG. 4A, Effect of FSK on cell size in INS-1 cells exposed to FSK. Effect of mTOR inhibitor PP242 shown. FIG. 4B, Effect of short term (2 hours) or long term (16 hours) FSK treatment on glycolytic flux, measured by lactate production (top), and on ATP generation (bottom) in primary cultured islets. (*; P<0.05. Data are means±s.d.) FIG. 4C, Effects of FSK pre-treatment (16 hours) on INS-1 cell survival following exposure to oxidative stress (150 μM H₂O₂). Cell viability monitored by trypan blue exclusion (top) and by immunoblot for cleaved caspase-3 protein amounts (bottom). Effect of rapamycin indicated. (*, P<0.05; **, P<0.01. Data are means±s.d.) FIG. 4D, Left, effect of rapamycin on HMOX1 mRNA amounts in pancreatic islets exposed to FSK. Right, immunoblot showing effect of FSK on HIF1α and phospho-S6 protein amounts in primary cultures of mouse pancreatic islets. FIG. 4E. and FIG. 4F. Immunohistochemical analysis showing effects of hepatic Ad-GLP-1 expression on S6 phosphorylation (FIG. 4E) and STZ-induced apoptosis (FIG. 4F) in pancreatic islets of ad libitum fed mice. Administration of rapamycin indicated.

FIG. 5: Q-PCR analysis of early (NR4A2) and late (AldoA, TPI, Glut 1, HMOX1) cAMP inducible genes identified in gene profiling assays of INS-1 cells exposed to FSK. Effect of FSK exposure for different times shown.

FIG. 6: Transient assay showing effect of FSK or IBMX on HRE-luciferase reporter activity in INS-1 cells. Co-incubation with PKA inhibitor H89 shown.

FIG. 7: Left, transient assay of a HIF-1α-luciferase translational reporter in INS-1 cells exposed to FSK and rapamycin as indicated. Fold-induction of HIF1α-luc or control luc vector activity in cells exposed to FSK versus unstimulated cells indicated. (*; P<0.05. Data are means±s.d.) Right, Q-PCR analysis of NR4A2 mRNA amounts in INS-1 cells exposed to FSK for 2 or 16 hours. Effect of rapamycin treatment shown.

FIG. 8: Left, immunoblot showing effects of FSK and DMOG on accumulation of HIF1α in primary hepatocytes. Treatment with rapamycin indicated. Right, transient assay of HRE-luc reporter activity in hepatocytes exposed to FSK and DMOG, alone or in combination.

FIG. 9: Left, immunoblot showing effect of A-CREB expression on IRS2-AKT signaling and HIF1α accumulation in INS-1 cells. Right, immunoblot showing effect of FSK on mobility of DEPTOR in INS-1 cells. Phospho- and dephospho-DEPTOR bands indicated. Co-incubation with PI3K inhibitor LY294002 as shown.

FIG. 10: Immunoblot showing effect of FSK on HIF1α accumulation and S6 phosphorylation. Co-treatment with ATP competitive mTOR inhibitor PP242 indicated.

FIG. 11: Circulating concentrations of GLP1 in mice expressing Adenoviral GLP-1 (GLP) or control Ad-GFP (control). Administration of Rapamycin or STZ indicated. GLP-1 levels were determined by Elisa Assay.

FIG. 12: Left, circulating glucose concentrations in control (Ad-GFP) and Ad-GLP1 expressing mice under ad libitum feeding conditions. Right, effects of STZ on glucose levels in mice expressing Ad-GLP-1 or control Ad-GFP.

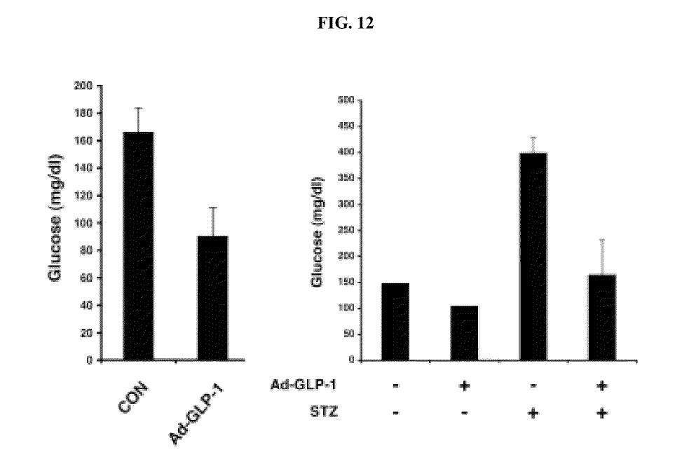

### DESCRIPTION OF ILLUSTRATIVE EMBODIMENTS

The present invention is based, in part, on the identification that GLP-1 can promote pancreatic cell viability by increasing the accumulation of hypoxia-inducible factor-1α (HIF i a) in beta cells via the mTOR pathway. Since iron chelators such as deferoxamine (DFO) or deferasirox can stabilize HIF 1α, these results indicate that the combination of a GLP-1 agonist and an iron chelator may be particularly beneficial for treating diabetes and/or promoting pancreatic islet cell survival.

Under feeding conditions, the incretin hormone GLP-1 promotes pancreatic islet viability by triggering the cAMP pathway in beta cells. Increases in PKA activity stimulate the phosphorylation of CREB, which in turn enhances beta cell survival by upregulating IRS2 expression. Although sustained GLP-1 action appears important for its salutary effects on islet function, the transient nature of CREB activation has pointed to the involvement of additional nuclear factors in this process. Following the acute induction of CREB-regulated genes, cAMP triggers a second delayed phase of gene expression that proceeds via the HIF transcription factor. Increases in cAMP promote the accumulation of HIF1α in beta cells by activating the mTOR pathway. As exposure to rapamycin disrupts GLP-1 effects on beta cell viability, these results demonstrate how a pathway associated with tumor growth also mediates salutary effects of an incretin hormone on pancreatic islet function. It is anticipated that, in certain embodiments, administering both an iron chelator and a GLP-1 agonist to a subject with diabetes may allow for lower concentrations of the GLP-1 agonist to be used to achieve a similar therapeutic effect.

### I. HIF1 PATHWAY

Current evidence indicates that HIF exerts a supportive role in islet function, although the mechanisms by which HIF activity are modulated in beta cells have not been addressed. Pancreatic islet expression of HIFβ/ARNT is decreased in human diabetes, for example (Gunton et al., 2005). Moreover, mice with a knockout of HIF-1α in islets display glucose intolerance and beta cell dysfunction, due in part to the decreased expression of glycolytic genes (Cheng et al.). Indeed, glycolytic flux has been found to play a key role in beta cell proliferation, providing a direct link between this HIF-regulated pathway and the maintenance of islet mass (Porat et al., 2011). The degree of HIF activation appears critical; however, unbridled HIF induction in mice with a knockout of the E3 ligase Von Hippel-Lindau (VHL) leads to persistent defects in glucose-stimulated insulin secretion (Cantley et al., 2009).

The below results indicate that GLP-1 promotes islet viability through the upregulation of HIF1α. GLP-1 stimulates HIF1α accumulation via the cAMP-mediated induction of the mTOR pathway in beta cells. cAMP appears to trigger mTOR activation in part via the CREB-mediated induction of IRS2-AKT signaling. Additional regulatory inputs appear likely, as the effects of cAMP on mTOR activity are cell-type dependent. Without wishing to be bound by any theory, it is proposed that PKA may regulate additional mTOR regulators that are selectively expressed in beta cells and perhaps other endocrine cell types. The identification of these factors should provide further insight into the mechanisms by which GLP-1 promotes pancreatic islet viability.

An iron chelator may be used to reduce the HIF-1α dysfunction observed in diabetes. In certain preferred embodiments, an iron chelator may be administered to a patient in combination with a GLP-1 agonist to treat diabetes, such as type II diabetes. Iron chelators that may be used with the present invention include deferoxamine and deferasirox. A HIF1 pathway agonist which is not an iron chelator, such as cAMP, may be used in certain embodiments of the present invention to promote HIF-1α activity. For example, in some embodiments, a cAMP agonist such as, e.g., Forskolin at a concentration of about 1-25 μM, about 5-20 μM, about 5-15 μM, about 10 uM, or any range derivable therein, may be included in a cell media to promote survival of beta cells in vitro.

Deferoxamine is a bacterial siderophore that has been used clinically for several decades to treat acute iron poisoning. In some embodiments, deferoxamine may be administered to a subject in an amount of about 100-1000 mg, and subsequent doses of about 500 mg may be administered every 4-12 hours. The total amount of administered should typically not exceed about 6000 mg in 24 hours. In some embodiments, an initial dose of about 1000 mg of deferoxamine is administered at a rate less than or equal to about 15 mg/kg/hr. Deferoxamine may be administered, e.g., intramuscularly, intravenously, or subcutaneously.

Deferoxamine may be administered subcutaneously, e.g., in combination with a GLP-1 agonist. In some embodiments, about 100-2000 mg may be administered s.c. daily (e.g., about 20-40 mg/kg/day). In some embodiments, about 100, 200, 300, 400, 500, 600, 700, 800, 900, 1000, 1100, 1200, 1300, 1400, 1500, 1600, 1700, 1800, 1900, or about 2000 mg, or any range derivable therein, of deferoxamine may be administered s.c. daily. A GLP-1 agonist and deferoxamine may be comprised in a pharmaceutical composition formulated for subcutaneous administration. Deferoxamine may be administered over an 8-24 hour period, e.g., via a small portable pump capable of providing continuous mini-infusion. The duration of infusion may be individualized.

Deferasirox was FDA approved in 2005 and has been clinically used to treat patients who are receiving long-term blood transfusions. Deferasirox is marketed under the brand name Exjade™ and is manufactured by Novartis (Basel, Switzerland). Deferasirox may be administered to a subject in an amount of about 5-30 mg/kg/day. In some embodiments, about 100, 200, 300, 400, 500, 600, 700, 800, 900, 1000, 1100, 1200, 1300, 1400, or about 1500 mg, or any range derivable therein, of deferasirox may be administered s.c. daily. Serum ferritin may be monitored, e.g., about once per month or every 2-5 weeks, and the dose of deferasirox may be adjusted if necessary, e.g., based on serum ferritin trends. Deferasirox is typically administered orally. Nonetheless, it is anticipated that deferasirox may in various embodiments be administered subcutaneously, intramuscularly, or intravenously. In some embodiments, deferasirox may be included in a pharmaceutical composition in combination with a GLP-1 agonist.

### II. GLP-1 AGONISTS

In certain embodiments, an agonist of glucagon-like peptide-1 (GLP-1) is administered to a patient in combination with an iron chelator. GLP-1 is derived from the transcription product of the proglucagon gene. GLP-1 is primarily produced by the intestinal L cell that secretes GLP-1 as a gut hormone. The biologically active forms of GLP-1 are: GLP-1-(7-37) and GLP-1-(7-36)NH2, which result from selective cleavage of the proglucagon molecule. In various embodiments, the GLP-1 agonist may be selected from the group consisting of exenatide, bydureon, liraglutide, albiglutide, taspoglutide, and lixisenatide.

The incretin hormone GLP-1 enhances islet cell survival through induction of the cAMP pathway in beta cells (Drucker, 2006; Drucker and Nauck, 2006). In turn, cAMP signaling stimulates the phosphorylation and activation of CREB, leading to increases in gene expression that promote beta cell viability. Disruption of CREB activity, through transgenic expression of dominant negative CREB inhibitors in beta cells, leads to hyperglycemia and diabetes, due in part to decreases in pancreatic islet mass and to consequent reductions in circulating insulin concentrations (Inada et al., 2004; Jhala et al., 2003).

CREB has been found to promote islet cell survival by upregulating insulin receptor substrate 2 (IRS2) gene expression and thereby activating the Ser/Thr kinase AKT. IRS2-AKT signaling appears critical for GLP-1 action; pancreatic islet cells from mice with a knockout of the IRS2 gene are resistant to growth promoting effects of GLP-1 agonist (Park et al., 2006). Conversely, IRS2 over-expression in islet cells appears sufficient to improve islet mass and glucose homeostasis in a mouse model of diabetes (Norquay et al., 2009).

GLP-1 stimulates the expression of IRS2 and other genes in part via the PKA-mediated phosphorylation of CREB at Ser133 (Altarejos and Montminy, 2011). CREB promotes target gene expression with burst-attenuation kinetics; rates of transcription peak within 1 hour and decrease to near baseline levels after 4 hours, paralleling the phosphorylation and subsequent dephosphorylation of CREB. By contrast with the transient kinetics of CREB activation, however, sustained GLP-1 action appears important for its salutary effects on islet function (Buse et al., 2010).

As shown in the below examples, GLP-1 promotes two phases of cAMP-dependent gene expression in beta cells; an acute CREB-mediated phase and a delayed phase that proceeds via the hypoxia inducible factor (HIF). GLP-1 was found to increase HIF activity through induction of the IRS2-AKT pathway and through the subsequent activation of the Ser/Thr kinase mTOR, a central regulator of cell growth and proliferation (Polak and Hall, 2009; Zoncu et al., 2011). Based on its ability to stimulate oxidative stress defense and metabolic programs that enhance beta cell viability, the data in the examples below support the idea that HIF can mediate long-term effects of GLP-1 on pancreatic islet function.

A. Exenatide

Exenatide is a GLP1 agonist that may be used to maintain blood glucose levels and treat aspects of diabetes. Exenatide is marketed as Byetta™ and manufactured by Amylin Pharmaceuticals and Eli Lilly and Company. Exenatide typically administered to a patient as a subcutaneous injection, e.g., of the abdomen, thigh, or arm. Exenatide is typically administered within about 1 hour before the first and last meal of the day.

In various aspects, exenatide has the sequence: H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2 (SEQ ID NO:1). Exenatide is typically supplied for subcutaneous injection as a sterile, preserved isotonic solution in a glass cartridge that has been assembled in a pen-injector (pen). Each milliliter (mL) may contain about 250 micrograms (mcg) of synthetic exenatide, about 2.2 mg metacresol as an antimicrobial preservative, mannitol as a tonicity-adjusting agent, and glacial acetic acid and sodium acetate trihydrate in water for injection as a buffering solution at pH 4.5. Prefilled pens may be used to deliver unit doses of 5 mcg or 10 mcg. Commercially available prefilled pens can typically deliver 60 doses to provide for 30 days of twice daily administration (BID). Although, in certain preferred embodiments, exenatide may be administered subcutaneously, it is nonetheless anticipated that exenatide may in certain embodiments be administered via another route, e.g., intravenous, intramuscular, etc.

Bydureon™ is an extended release version of exenatide that may be used in various embodiments of the present invention. Bydureon™ may be administered to a subject less frequently than Byetta™. For example, bydureon may be administered subcutaneously to a subject about once per week. Bydureon™ is commercially available from Amylin Pharmaceuticals, Inc. (San Diego, Calif.).

B. Liraglutide

Liraglutide (NN2211) is a long-acting GLP-1 analog that may be administered to a subject for the treatment of type 2 diabetes. Liraglutide is a DPP-IV-resistant GLP-1 analog that has been modified by 2 amino acid changes, i.e., one addition and one substitution, and by the addition of a fatty acid group that enables it to form a noncovalent bond with serum albumin following SC administration, thus reducing its renal clearance and increasing its PK profile. The half-life of liraglutide in humans is approximately 12 hours and may require only 1 injection per day. Liraglutide marketed under the brand name Victoza™ and is manufactured by Novo Nordisk.

In various aspects, liraglutide has the chemical formula: L-histidyl-L-alanyl-L-α-glutamylglycyl-L-threonyl-L-phenylalanyl-L-threonyl-L-seryl-L-α-aspartyl-L-valyl-L-seryl-L-seryl-L-tyrosyl-L-leucyl-L-α-glutamylglycyl-L-glutaminyl-L-alanyl-L-alanyl-N6-[N-(1-oxohexadecyl)-L-γ-glutamyl]-L-lysyl-L-α-glutamyl-L-phenylalanyl-L-isoleucyl-L-alanyl-L-tryptophyl-L-leucyl-L-valyl-L-arginylglycyl-L-arginyl-glycine.

Liraglutide typically has a half-life after subcutaneous injection of about 11-15 hours after subcutaneous injection, making it suitable for once-daily dosing (less frequent than the currently approved Byetta™ form of exenatide, which is twice daily, but considerably more frequent than the once weekly Bydureon™ form of exenatide). Although, in certain embodiments, liraglutide is administered subcutaneously, it is nonetheless anticipated that exenatide may in certain embodiments be administered via another route, e.g., intravenous, intramuscular, etc.

C. Ablugtide

In some embodiments, the GLP-1 agonist may be albiglutide. The long-acting GLP-1 receptor agonist albiglutide is a recombinant human serum albumin (HSA)-GLP-1 hybrid protein, i.e., a dipeptidyl peptidase-4-resistant glucagon-like peptide-1 dimer fused to human albumin. As the GLP-1 epitopes are fused to the larger HSA molecule, albiglutide exhibits a pharmacokinetic profile resembling that of albumin in the circulation. Albiglutide is currently being investigated by GlaxoSmithKline for treatment of type 2 diabetes. Albiglutide may have a half-life of about four to seven days after administration (Matthews et al. (2008) J. Clin. Endocrinol. Metab. 93 (12): 4810-4817).

D. Taspoglutide

The GLP-1 agonist may be taspoglutide (R1583). Taspoglutide a glucagon-like peptide-1 analog that is the 8-(2-methylalanine)-35-(2-methylalanine)-36-L-argininamide derivative of the amino acid sequence 7-36 of human glucagon-like peptide I.

In various aspects, taspoglutide has the chemical formula: H2N-His-2-methyl-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-2-methyl-Ala-Arg-CONH2 (SEQ ID NO:2). Taspoglutide is a long-acting GLP-1 analog in which amino acids 8 and 35 of the native GLP-1 peptide are substituted with aminoisobutyric acid to prevent DPP-IV and protease-mediated cleavage at the N- and C-terminus, respectively. R1583 may be formulated as a zinc-based drug to prolong its PK activity. Various dosages of taspoglutide may be administered to a patient, e.g., 1-30 mg s.c. Taspoglutide is manufactured by Ipsen and Roche. Taspoglutide is further described in Nauck et al. (2009) Diabetes Care. 32(7):1237-43, which is herein incorporated by reference in its entirety.

E. Lixisenatide

Lixisenatide (AVE0010) is a GLP-1 agonist that may be used with the present invention. Lixisenatide is an exendin-4-based GLP-1 receptor agonist that exhibits approximately 4-fold greater affinity for the human GLP-1 receptor compared with native GLP-1. Lixisenatide may be administered to a subject, e.g., once or twice a day. In some embodiments, metformin and/or SU therapy may be administered in combination with a GLP-1 agonist such as, e.g., Lixisenatide. Lixisenatide may be administered at a dosage of, e.g., about 5-20 micrograms (mcg)/injection. The half-life of AVE0010 may range from about 2.5 to 4 hours. Clinical trials have indicated that lixisenatide can significantly improve glycaemic control in mildly hyperglycaemic patients with Type 2 diabetes on metformin (Ratner et al. (2010) Diabet Med. September; 27(9):1024-32).

### III. COMBINATION THERAPIES

In various aspects of the present invention, a GLP-1 agonist may be administered to a subject in combination with an iron chelator. The GLP-1 agonist may be administered substantially simultaneously with the iron chelator, e.g., in a single pharmaceutical preparation, to a subject in an amount effective to treat diabetes. Alternately, the GLP-1 agonist and iron chelator may be administered sequentially to a subject in an amount effective to treat diabetes. In some embodiments, the GLP-1 agonist and the iron chelator are present in an amount sufficient to synergistically treat diabetes, such as type II diabetes, in a human subject.

More generally, the GLP-1 agonist and the iron chelator may be provided in a combined amount effective to treat or reduce one or more symptoms of diabetes or produce a therapeutic benefit for the treatment of diabetes. This process may involve contacting the cell(s) with a GLP-1 agonist and an iron chelator at the same time or within a period of time wherein separate administration of the GLP-1 agonist and iron chelator to a cell, tissue or organism produces a desired therapeutic benefit. This may be achieved by contacting the cell, tissue or organism with a single composition or pharmacological formulation that includes both a GLP-1 agonist and an iron chelator, or by contacting the cell with two or more distinct compositions or formulations, wherein one composition includes a GLP-1 agonist and the other includes and an iron chelator.

The terms “contacted” and “exposed,” when applied to a cell, tissue or organism, are used herein to describe the process by which a GLP-1 agonist and an iron chelator are delivered to a target cell (e.g., a beta cell), tissue or organism or are placed in direct juxtaposition with the target cell, tissue or organism. To achieve reducing effects of diabetes, e.g., reducing one or more symptoms associated with diabetes, such as type II diabetes, the GLP-1 agonist and iron chelator are delivered to one or more cells in a combined amount effective to result in a therapeutic effect (e.g., promote survival of beta cells, promote insulin release or sensitivity of cells, reduce glucagon secretion).

The GLP-1 agonist may precede, be co-current with and/or follow the iron chelator by intervals ranging from minutes to weeks. In embodiments where the GLP-1 agonist and iron chelator are applied separately to a cell, tissue or organism, one would generally ensure that a significant period of time did not expire between the time of each delivery, such that the GLP-1 agonist and iron chelator would still be able to exert an advantageously combined effect on the cell, tissue or organism. In other aspects, the iron chelator may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4 weeks or more, and any range derivable therein, prior to and/or after administering the GLP-1 agonist.

Various combination regimens of the GLP-1 agonist and iron chelator may be employed. Non-limiting examples of such combinations are shown below, wherein a composition GLP-1 agonist is “A” and the iron chelator is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of the GLP-1 agonist and iron chelator to a cell, tissue or organism may follow general protocols for the administration of the compounds, taking into account the toxicity, if any. It is expected that the treatment cycles would be repeated as necessary. In particular embodiments, it is contemplated that various additional agents, such as therapeutics to treat diabetes or a complication resulting from type II diabetes, may be applied in any combination with the present invention.

### IV. PHARMACEUTICAL PREPARATIONS

Pharmaceutical compositions of the present invention comprise an effective amount of a GLP-1 agonist and an iron chelator dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains a GLP-1 agonist and an iron chelator will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The GLP-1 agonist and iron chelator may comprise different types of carriers depending on whether it is to be administered in solid, liquid or aerosol form, and whether it need to be sterile for such routes of administration as injection. In certain preferred embodiments, the GLP-1 agonist, with or without iron chelator, is administered subcutaneously. Nonetheless, it is anticipated that the present invention may be administered intravenously, intradermally, transdermally, intrathecally, intraarterially, intramuscularly, subcutaneously, mucosally, orally, inhalation (e.g., aerosol inhalation), injection, infusion, continuous infusion, via a catheter, via a lavage, in lipid compositions (e.g., liposomes), or by other method or any combination of the forgoing as would be known to one of ordinary skill in the art (see, for example, Remington: The Science and Practice of Pharmacy, 21^{st} edition, Pharmaceutical Press, 2011, incorporated herein by reference).

The GLP-1 agonist and iron chelator may be formulated into a composition in a free base, neutral or salt form. Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In some embodiments, the composition may be combined or mixed thoroughly with a semi-solid or solid carrier. The mixing can be carried out in any convenient manner such as grinding. Stabilizing agents can be also added in the mixing process in order to protect the composition from loss of therapeutic activity. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include GLP-1 agonist and iron chelator, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the GLP-1 agonist and iron chelator may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In further embodiments, a pharmaceutical preparation comprising a GLP-1 agonist and an iron chelator may be administered via a parenteral route, such as via subcutaneous injection. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions can be prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Immunoblot and immunoprecipitation: Immunoblots were performed as described (Dentin et al., 2008). For immunoprecipitations, cell lysates were incubated with primary antibody and 25 μl of a 50% slurry of protein G agarose beads for 4 hours with rotation at 4° C. Immunoprecipitates were washed with lysis buffer and denatured by boiling in 20 μl SDS sample buffer.

Immunofluorescence:

Whole pancreases were fixed in 4% paraformaldehyde, fresh-frozen, and cryosectioned. Sections were incubated with primary antibody overnight and with fluorophore conjugated secondary antibody for 1 hour. Sections were mounted with Vectashield mounting medium containing DAPI (Vector Labs) and analyzed in a Zeiss LSM 710 Laser Scanning Confocal Microscope. TUNEL staining on paraffin embedded pancreatic sections was performed with the ApoBrdU DNA Fragmentation Assay Kit (BioVision; K401-60).

Chromatin Immunoprecipitation:

INS-1 cells were plated on 15 cm dishes and near-confluent cells were exposed to forskolin as specified. Chromatin immunoprecipitation with HIF-1α and CRTC2 antisera was performed as described (Ravnskjaer et al., 2007).

Cell Culture and Transfection:

INS-1 insulinoma cells were cultured in RPMI 1640 (Mediatech) with 10% heat inactivated fetal bovine serum (Sigma), 2 mM glutamine (Mediatech), 1 mM sodium pyruvate (Mediatech), 100 μg/ml penicillin-streptomycin (Mediatech) and 0.05 mM β-mercaptoethanol. HEK 293T cells were cultured in DMEM (Mediatech) with 10% fetal bovine serum and 100 μg/ml penicillin-streptomycin. Forskolin (10 μM), LY294002 (50 μM), and DMOG (1 mM) were added to cells as indicated. Cells were exposed to rapamycin (50 nM) or to cycloheximide (100 μg/ml) overnight. For glucose and Exendin-4 treatments, INS-1 cells were glucose and serum starved for 1 hour, and stimulated with glucose or 2-deoxyglucose (20 mM) with or without Exendin-4 (10 nM). HEK 293T cells were transfected with polyethylenimine (PEI) and INS-1 cells with Lipofectamine2000 (Invitrogen; 11668-019). For HIF-1α translational reporter experiment, INS-1 cells were transfected with pLUXHIF1α5′UTR or pLUX control vector (generous gift of John Blenis). After 24 hours, cells were exposed to rapamycin overnight where indicated. After forskolin treatment, cells were harvested for Renilla/firefly luciferase activity. Normalized luciferase activities are shown.

Plasmids and DNA Manipulations:

Site-directed mutagenesis was performed with Pfu Turbo polymerase (Stratagene). For adenovirus construction, cDNAs and short hairpin sequences were subcloned in the pAdTRACK vector. Complete viral vectors were generated by homologous recombination with the AdEASY vector as described (Koo et al., 2005).

Isolation of Primary Islets:

Pancreatic islets were isolated as described (Jhala et al., 2003). Primary islets were cultured in complete RPMI medium for 1-2 days to recover from the isolation.

Lactate, ATP and Oxidative Stress Measurements:

Lactate release was measured using a lactate assay kit from Eton Bioscience (1200012002).

Intra-islet ATP concentration was measured using the ATP Bioluminescence Assay Kit HS II (Roche, 11 699 709 001). For viability measurements, INS-1 cells were treated with forskolin and rapamycin for 16 hours. After exposure to 150 μM hydrogen peroxide, cells were trypsinized and harvested, and viability was assessed by trypan blue dye exclusion.

Cell Size Determination:

For cell size measurements, INS-1 cells were plated on 6 cm dishes, treated as indicated, trypsinized and resuspended in PBS. Cell diameter was measured using a particle size counter.

Gene Profiling Experiment:

Gene profiling experiments were performed on total RNA from INS-1 cells using an Affymetrix Rat Gene Array as previously described (Zhang et al., 2005).

Adenoviral GLP-1 Delivery and Streptozotocin-Induced Diabetes:

Ad-GLP1 virus was provided by Dr. G. Parsons (Parsons et al., 2007). GLP-1 expressing or control (Ad-GFP) virus was delivered to male C57BL/6J mice (The Jackson Laboratory; 000664) by tail vein injection. Streptozotocin was injected at 100 mg/kg in citrate buffer once a day for 3 consecutive days. Rapamycin was injected every other day at 10 mg/kg body in PBS containing 5% Tween80 and 5% PEG400.

Antibodies and Reagents:

Reagents used in this study were obtained from the following sources: Rabbit polyclonal antibody to HIF-1α from Cayman Chemical (10006421); rabbit polyclonal antibody to HSP90 from Santa Cruz Biotechnology (sc-79470); mouse monoclonal antibody to tubulin from Millipore (05-829); rabbit polyclonal antiserum to phospho (S877) RAPTOR from Millipore; anti-FLAG M2 agarose from Sigma (A2220); rabbit polyclonal antibodies to phospho-S6 (Cell Signaling; 4857); rabbit monoclonal antibody to cleaved caspase-3 (Cell Signaling; 9664); phospho-Akt (T308) (Cell Signaling; 4056); phospho-Akt (S473) (Cell Signaling; 9271); phospho-PKA substrate (RRXpS/T) (Cell Signaling; 9621); mouse monoclonal antibody to S6 (Cell Signaling; 2317); phospho (T1462) TSC2 (Cell Signaling; 3617); guinea pig polyclonal antibody to insulin (Zymed; 180067). Dimethyloxalylglycine (DMOG) from Frontier Scientific (D1070); rapamycin from LC laboratories (R-5000); forskolin from Calbiochem; PP242 from Sigma (P0037); trypan blue solution from Sigma (T8154); LY294002 from Cell Signaling Technology (9901); H89 from Calbiochem (371963); streptozotocin from Sigma (S0130); cycloheximide from Calbiochem (239674).

Plasmids expressing cDNAs for HA-HIF-1α P402A/P564A (18955) were purchased from Addgene. HRE-LUC reporter construct was generously provided by Randall Johnson (UCSD).

RNAi-adenoviruses were constructed expressing U6 promoter driven short hairpin RNAs directed against mouse and rat HIF-1α (GGGCAGTCAATGGATGAGAGTG, SEQ ID NO:3) cDNAs.

### Oligonucleotides Used for ChIP Analysis:

| Target enhancer | | |
|---|---|---|
| region (rat) | | |
| NR4A2 | Fw | 5′-GCGCAGACTTTAGGTGCATG-3′ (SEQ ID NO: 4) |
| | Rev | 5′-TGTTTATGTGGCTCGCGCTG-3′ (SEQ ID NO: 5) |
| GLUT1 | Fw | 5′-ACAGGCGTGCTGGCTGACAC-3′ (SEQ ID NO: 6) |
| | Rev | 5′-TGATGATTCGGGCAAGTGCC-3′ (SEQ ID NO: 7) |
| HMOX1 | Fw | 5′-TGGCAAGAAGGAGAGCGGAC-3′ (SEQ ID NO: 8) |
| | Rev | 5′-GTCCACAGAAGGAACGTGTC-3′ (SEQ ID NO: 9) |

Real-Time Quantitative PCR:

mRNA levels were quantified by Q-PCR analysis as previously described (Dentin, 2007). Oligonucleotides were designed to target rat gene sequences.

### Oligonucleotides Used for Q-PCR Analysis:

| Target | | |
|---|---|---|
| cDNA | | Sequence |
| Actin | Fw | 5′-TCTACAATGAGCTGCGTGTG-3′ (SEQ ID NO: 10) |
| | Rev | 5′-GGTCTCAAACATGATCTGGG-3′(SEQ ID NO: 11) |
| L32 | Fw | 5′-GAAAACCAAGCACATGCTGC-3′ (SEQ ID NO: 12) |
| | Rev | 5′-TTGTTGCACATCAGCAGCAC-3′ (SEQ ID NO: 13) |
| NR4A2 | Fw | 5′-CTACCTGTCCAAACTGTTGG-3′ (SEQ ID NO: 14) |
| | Rev | 5′-GGTAAGGTGTCCAGGAAAAG-3′(SEQ ID NO: 15) |
| IRS2 | Fw | 5′-TCTCCCAAAGTGGCCTACAA-3′(SEQ ID NO: 16) |
| | Rev | 5′-TCATGGGCATGTAGCCATCA-3′ (SEQ ID NO: 17) |
| ATF3 | Fw | 5′-AAGGAAGAGCTGAGATTCGC-3′ (SEQ ID NO: 18) |
| | Rev | 5′-CTCAGACTTGGTGACTGACA-3′ (SEQ ID NO: 19) |
| CRY2 | Fw | 5′-GAAGCAGATCTACCAACAGC-3′ (SEQ ID NO: 20) |
| | Rev | 5′-CACAGGGTGACTGAGGTCTT-3′ (SEQ ID NO: 21) |
| HIF-1α | Fw | 5′-ACCACTGCTAAGGCATCAGC-3′ (SEQ ID NO: 22) |
| | Rev | 5′-GCTCCTTGGATGAGCTTTGT-3′ (SEQ ID NO: 23) |
| GLUT1 | Fw | 5′-GCTTATGGGTTTCTCCAAACT-3′ (SEQ ID NO: 24) |
| | Rev | 5′-GTGACACCTCCCCCACATAC-3′ (SEQ ID NO: 25) |
| HMOX1 | Fw | 5′-AGGCTTTAAGCTGGTGATGG-3′ (SEQ ID NO: 26) |
| | Rev | 5′-ATACCAGAAGGCCATGTCCT-3′ (SEQ ID NO: 27) |
| Aldolase A | Fw | 5′-GAAGAAGGAGAACCTGAAGG-3′ (SEQ ID NO: 28) |
| | Rev | 5′-ACAGAGATTCACTGGCTGCG-3′ (SEQ ID NO: 29) |
| TPI1 | Fw | 5′-AGGAAGTACACGAGAAGCTC-3′ (SEQ ID NO: 30) |
| | Rev | 5′-CTCCAGTCACAGAACCTCCA-3′ (SEQ ID NO: 31) |
| PGK1 | Fw | 5′-GACTGTGGTACTGAGAGCAG-3′ (SEQ ID NO: 32) |
| | Rev | 5′-CCTGGCAAAGGCTTCCCATT-3′ (SEQ ID NO: 33) |
| PDK1 | Fw | 5′-CTGAGGAAGATCGACAGACT-3′ (SEQ ID NO: 34) |
| | Rev | 5′-GATATGGGCAATCCGTAACC-3′ (SEQ ID NO: 35) |
| LDHA | Fw | 5′-GTGCATCCCATTTCCACCAT-3′ (SEQ ID NO: 36) |
| | Rev | 5′-GAGTCAGTGTCACCTTCACA-3′ (SEQ ID NO: 37) |
| BNIP3 | Fw | 5′-GCGCACAGCTACTCTCAGCA-3′ (SEQ ID NO: 38) |
| | Rev | 5′-GTCAGACGCCTTCCAATGTAG-3′ (SEQ ID NO: 39) |

Lactate and ATP Measurement:

For lactate measurements, batches of 50 size-matched primary islets were cultured in RPMI medium without phenol red. After the indicated forskolin exposures, the islets were placed in fresh medium and incubated for 4 hours at 37° C. For ATP measurements, batches of 50 size matched primary islets were exposed to forskolin, harvested, and boiled in 200 μl Tris-HCl pH 7.75, 4 mM EDTA for four minutes.

### Example 2

### mTOR Links Incretin Signaling to HIF Induction in Pancreatic Beta Cells cAMP Stimulates CREB and HIF Pathways in β Cells

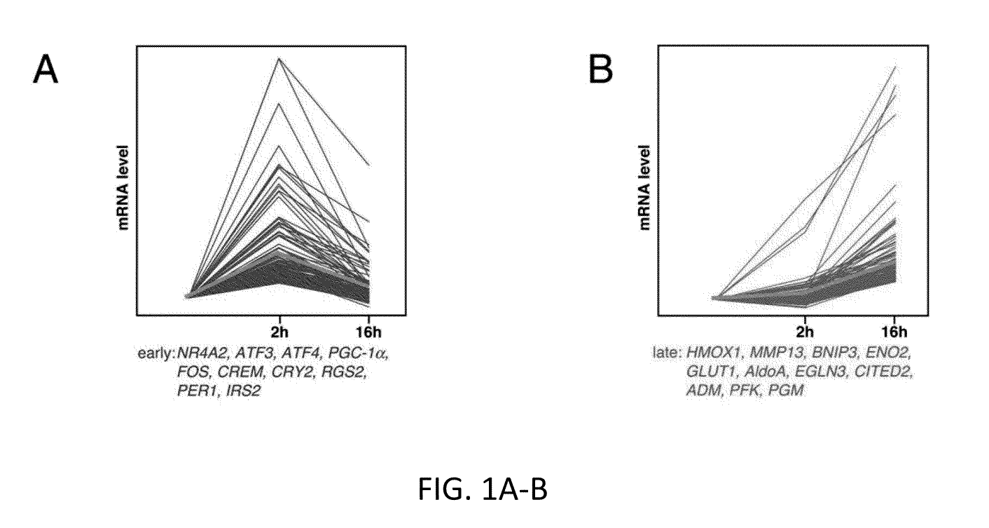

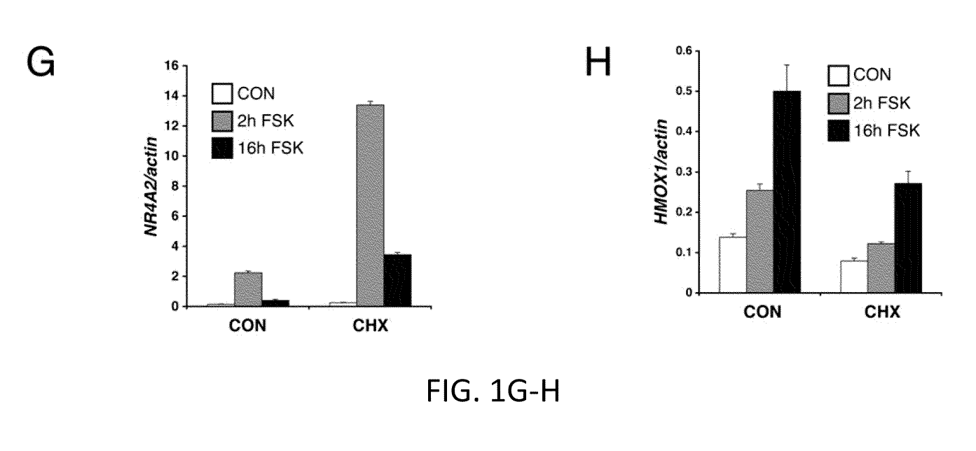

Gene profiling studies were performed to evaluate effects of the GLP-1-cAMP pathway in INS-1 insulinoma cells. Short-term (2 hour) exposure to the cAMP agonist Forskolin (FSK) up-regulated a number of CREB target genes that contain a conserved cAMP response element (CRE) and that are CREB-occupied in vivo (IRS2, RGS2, PGC1, NR4A2) (Zhang et al., 2005); these were down-regulated after prolonged (16 hour) treatment with FSK (FIG. 1A, FIG. 5).

A second set of genes were detected that were expressed at low levels at 2 hours, but increased after 16 hours exposure to FSK, when CREB target genes are down-regulated (FIG. 1B, FIG. 5). Many of these late response genes correspond to hypoxia-inducible genes by GO analysis; they code for proteins that promote glucose uptake and glycolysis (GLUT1, AldoA, TPI, PGK1) as well as oxidative stress defense (HMOX1). Similar to its effects in INS-1 cells, exposure to FSK stimulated two waves of gene expression in cultured mouse pancreatic islets, with CREB target genes predominating at early times followed by hypoxia-inducible genes at later times (FIG. 1C, FIG. 1D).

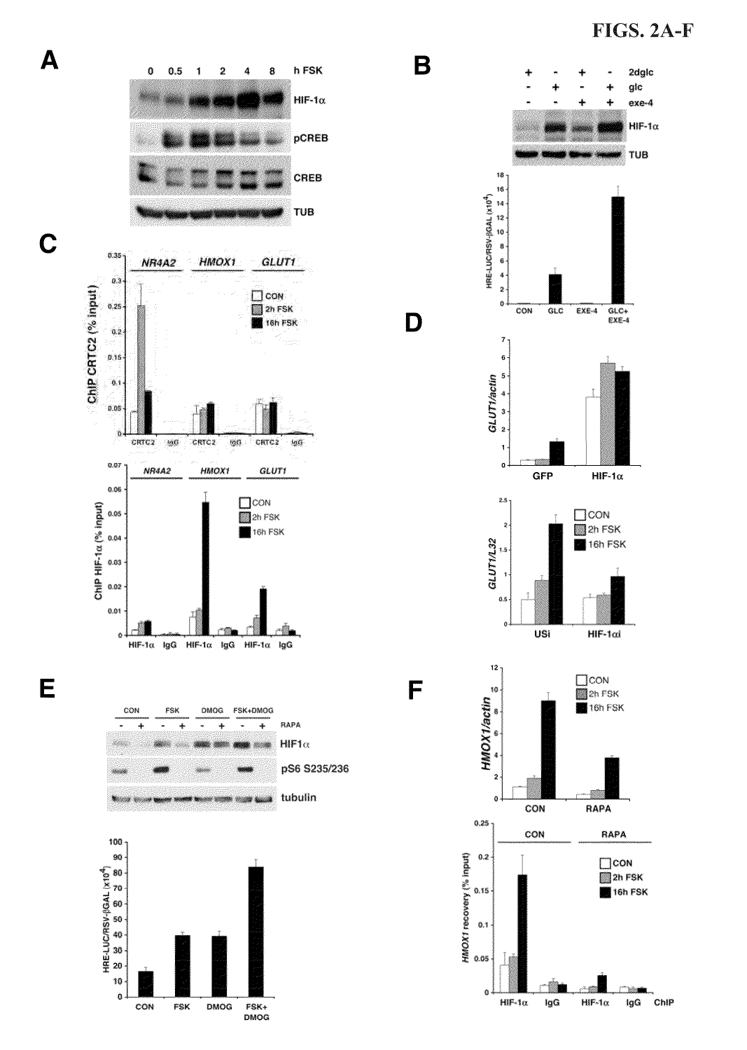

Based on these profiles, it was hypothesized that cAMP mediates induction of two distinct transcription pathways in beta cells. Supporting this notion, exposure to FSK increased the activity of a CRE-Luc reporter in INS-1 cells after 6 hours, but less so after 20 hours (FIG. 1E). By contrast, the activity of a hypoxia-inducible factor (HIF) reporter (HRE-luc) was low at early times (6 hours), but increased after 20 hours (FIG. 1F). Consistent with a role for cAMP in this process, exposure to phospho-diesterase inhibitor IBMX also increased HRE-luc reporter activity in INS-1 cells (FIG. 6). The effects of FSK and IBMX on HIF activity appear to be PKA-dependent because exposure to the PKA antagonist H89 blocked induction of the HRE-luc reporter by FSK as well as IBMX (FIG. 6).

The inventors hypothesized that the two phases of cAMP inducible transcription in beta cells may have different requirements for new protein synthesis. Supporting this idea, treatment with the protein synthesis inhibitor cycloheximide (CHX) enhanced the expression of CREB target genes, but it reduced the expression of late phase genes like HMOX1 by FSK (FIG. 1G, FIG. 1H).

GLP-1 Stimulates HIF Activity via Induction of mTOR

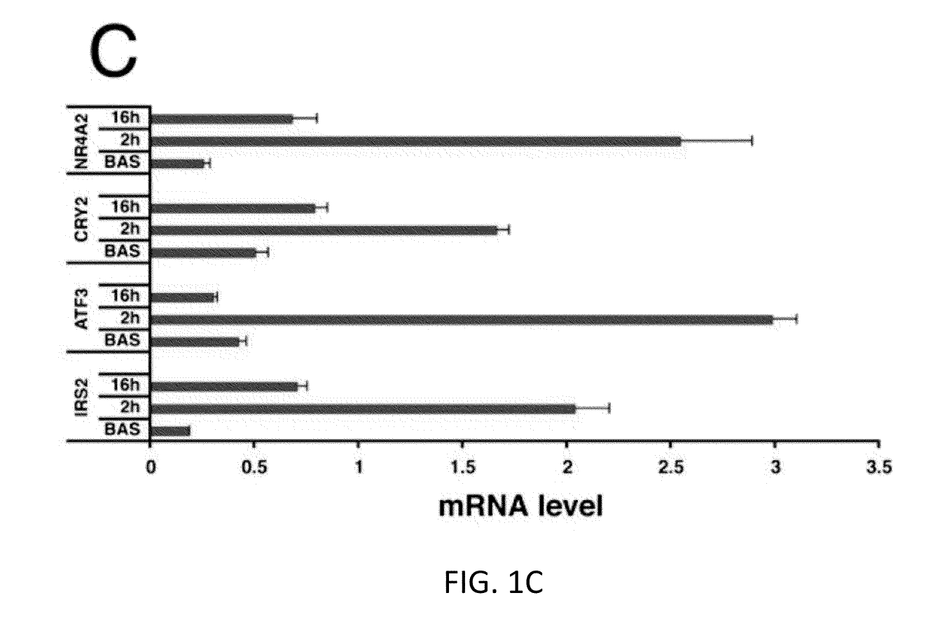

Originally identified as an oxygen-sensing heterodimer that contains an unstable alpha subunit and a constitutively expressed beta subunit, HIF induces metabolic reprogramming in response to hypoxia and growth factors signaling (Semenza, 2010). Hypoxia has been shown to increase HIF1α stability, whereas growth factors appear to increase the translation of HIF1αmRNA (Sengupta et al., 2010). Exposure of INS-1 cells to FSK increased HIF1α protein but not mRNA levels to maximal levels after 4-8 hours (FIG. 2A, FIG. 5). Similar to FSK, prolonged exposure to GLP-1 agonist (Exendin-4) also increased HIF1α protein accumulation and HRE-luc reporter activity in INS-1 cells, particularly under high glucose (20 mM) conditions, when GLP-1 is active (FIG. 2B).

By contrast with the delayed induction of HIF, FSK exposure promptly increased CREB phosphorylation after only 1 hour, returning to near baseline after 4 hours (FIG. 2A). Indeed, short-term treatment also enhanced recruitment of the CREB coactivator CRTC2 (Screaton et al., 2004) to early (NR4A2) but not late phase (HMOX1, GLUT1) genes by chromatin immunoprecipitation (ChIP) assay (FIG. 2C). Conversely, prolonged exposure to FSK increased the occupancy of HIF1α over late (HMOX1 and GLUT1) but not early (NR4A2) promoters.

Tests were performed to determine whether the slow accumulation of HIF1αprotein accounts for the delayed kinetics of HIF target gene expression in INS-1 cells. Supporting this idea, over-expression of HIF1α upregulated HIF target gene expression even in the absence of cAMP agonist, while RNAi mediated knockdown of HIF1α blocked it (FIG. 2D). Collectively, these studies indicate that activation of the cAMP pathway by GLP-1 triggers early and late phases of gene expression, which are coordinated by CREB and HIF, respectively.

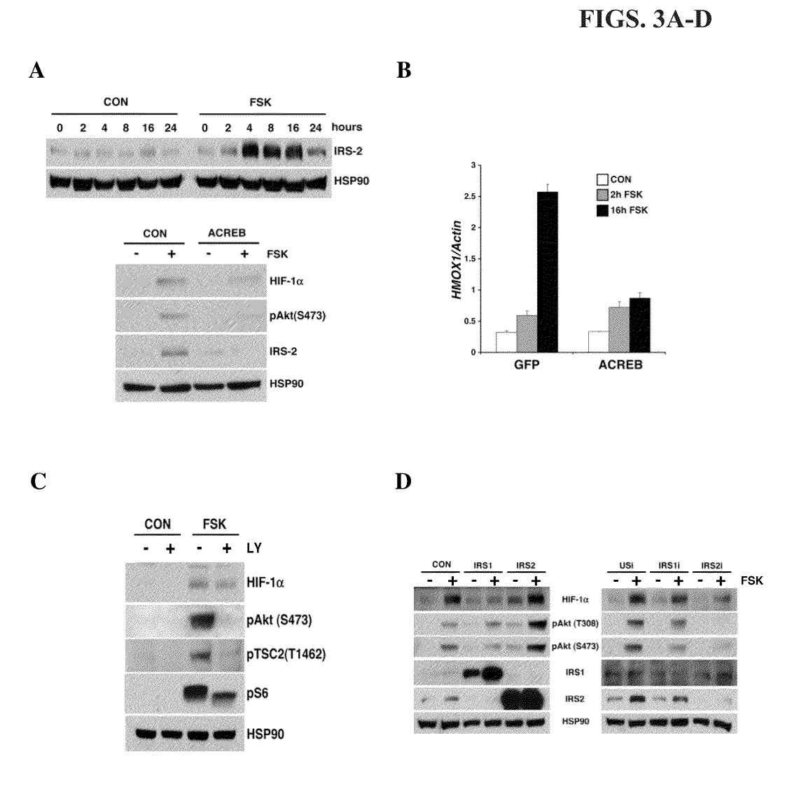

Based on the ability for mTOR to stimulate HIF1α translation in response to nutrient and growth factor signals (Sengupta et al., 2010), the inventors examined whether cAMP activates this pathway in beta cells. Exposure of INS-1 cells to FSK increased the phosphorylation of the ribosomal protein S6, a downstream target of the mTORC1 complex, which contains mTOR, mLST8, PRAS40, and Raptor (FIG. 2E). Consistent with the proposed role of mTORC1, FSK treatment increased the activity of a HIF1α-luc translational reporter (Choo et al., 2008) containing the 5′UTR of HIF1α (FIG. 7); these effects were blocked by co-treatment with the mTORC1 inhibitor rapamycin.

Exposure to rapamycin also disrupted S6 phosphorylation and HIF1α accumulation in INS-1 cells exposed to FSK (FIG. 2E). By contrast, triggering of the hypoxia pathway with the prolyl hydroxylase inhibitor Dimethyloxalyl Glycine (DMOG) increased HIF1α but not phospho-S6 protein amounts in INS-1 cells; and rapamycin did not attenuate DMOG-induced HIF1α accumulation (FIG. 2E). Rather, co-stimulation with DMOG and FSK increased HRE-luc reporter activity additively, suggesting that cAMP and hypoxia pathways stimulate HIF1α via distinct mechanisms (FIG. 2E).

The inventors tested whether the cAMP-dependent induction of late phase genes in islet cells proceeds via mTORC1. Consistent with its inhibitory effects on HIF1α accumulation, exposure to rapamycin blocked the recruitment of HIF1α to the HMOX1 promoter by ChIP assay of INS-1 cells (FIG. 2F). Correspondingly, rapamycin also attenuated the FSK-dependent upregulation of HMOX1 mRNA, but it had no effect on the induction of CREB target genes such as NR4A2 (FIG. 7). These results suggest that mTOR mediates the effects of cAMP on induction of late but not early phase genes in INS-1 cells.

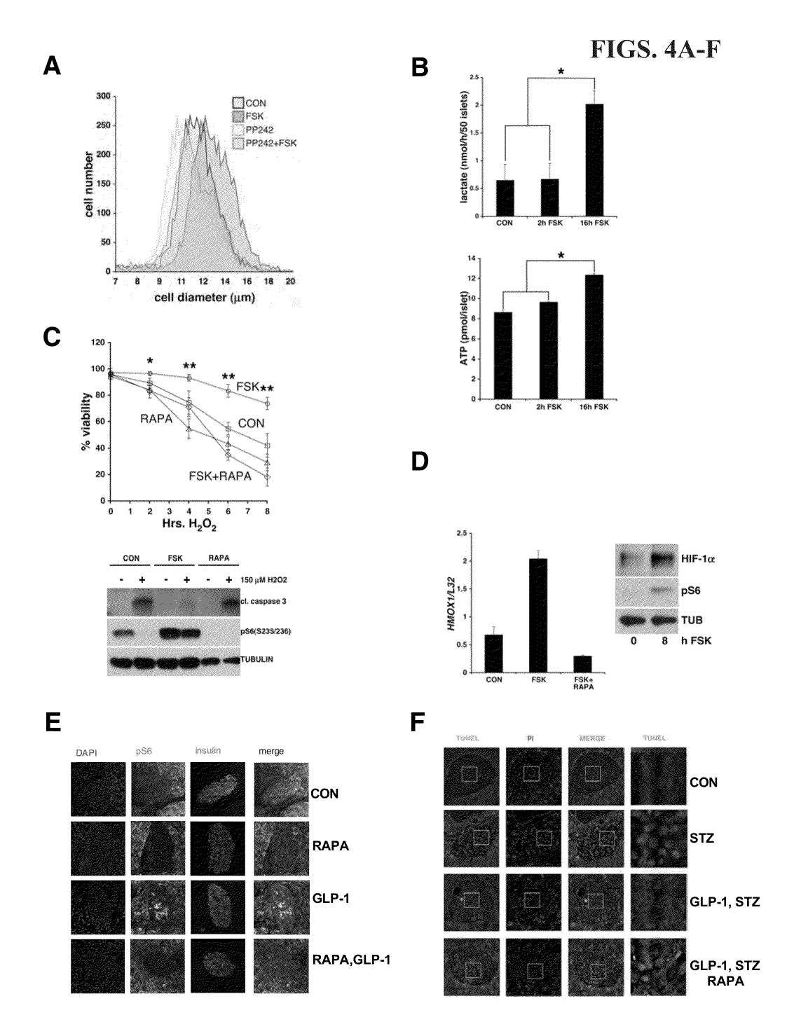

Although cAMP functions as a senescence signal in most cells, it promotes the growth and proliferation of a small subset of endocrine cells (Mantovani et al., 2005; Ringel et al., 1996), prompting us to test whether the effects of cAMP on HIF activity are cell context-dependent. By contrast with its stimulatory effects in INS-1 cells, exposure to FSK had no effect on HIF1α accumulation or HRE-luc reporter activation in cultured primary hepatocytes (FIG. 8). Arguing against general differences in HIF1α inducibility, exposure to DMOG triggered HIF1α accumulation and HRE reporter activation comparably in hepatocytes and INS-1 cells (FIG. 8). Taken together, these results indicate that the effects of cAMP on the HIF pathway are indeed cell-type restricted.

cAMP Promotes mTOR Activation via Induction of IRS2-AKT Signaling

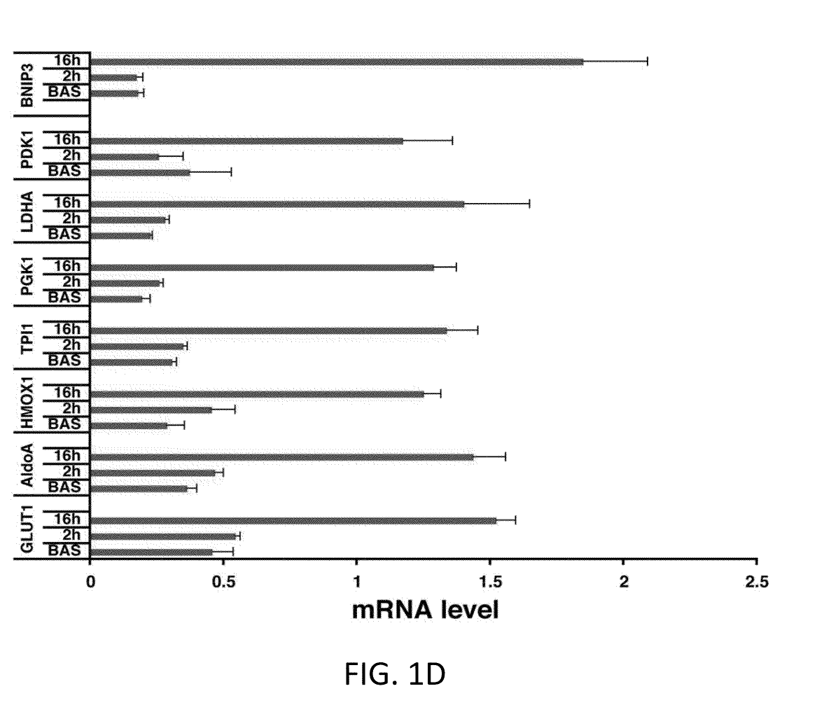

Realizing that the Ser/Thr kinase AKT stimulates mTOR activity and that CREB upregulates IRS2 expression in beta cells (Jhala et al., 2003), the inventors wondered whether cAMP enhances mTOR activity in part via induction of the IRS2-AKT pathway. Exposure to FSK triggered IRS2 accumulation as well as AKT activation in INS-1 cells (FIG. 3A). Disrupting CREB activity, by over-expression of the CREB inhibitor A-CREB, blocked IRS2-AKT pathway activation and HIF1α accumulation as well as HIF target gene (HMOX1) expression (FIG. 3A, FIG. 3B).

AKT has been found to enhance mTOR activity by phosphorylating the GTPase activating protein TSC2, an inhibitor of the small G protein Rheb, an mTOR activator (Zoncu et al., 2011). Amounts of phospho (Thr 1462) TSC2 were upregulated in INS-1 cells exposed to FSK (FIG. 3C); these effects were blocked when AKT activity was inhibited by co-incubation with the PI3 kinase inhibitor LY294002 (LY). The phosphorylation of TSC2 in INS-1 cells appears important for the subsequent induction of mTORC1 by FSK because treatment with LY inhibitor also reduced phospho-S6 protein amounts in cells exposed to FSK (FIG. 3C).

If IRS2 regulates mTOR activity, then altering cellular IRS2 protein amounts should correspondingly modulate HIF l a protein levels. Over-expression of IRS2 but not IRS1 in INS-1 cells potentiated AKT activity and HIF1α accumulation (FIG. 3D). Conversely, RNAi-mediated knockdown of IRS2 but not IRS1 decreased effects of FSK on the upregulation of AKT and HIF1α. Collectively, these results indicate that the acute activation of the IRS2-AKT pathway by CREB is required for subsequent induction of the HIF pathway by mTOR in response to cAMP signaling.

### mTOR-HIF Pathway Mediates GLP-1 Effects on β Cell Viability

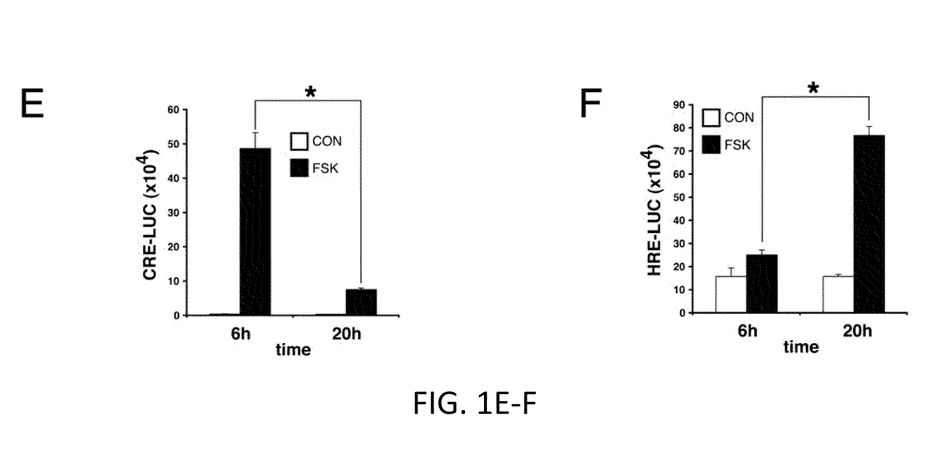

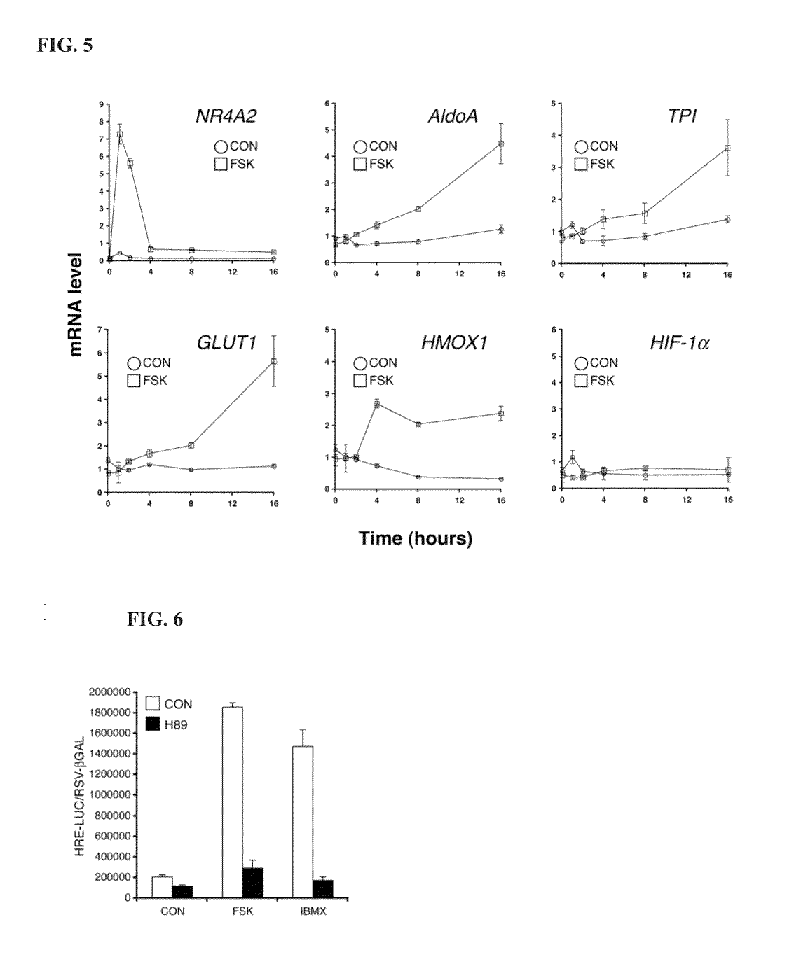

Realizing that cAMP stimulates mTOR activity and that mTOR regulates beta cell size (Granot et al., 2009; Ruvinsky et al., 2005), the inventors considered whether cAMP promotes beta cell growth via this pathway. Supporting this notion, FSK treatment increased the average diameter of INS-1 cells but not HEK293T cells (FIG. 4A; not shown); co-incubation with a direct inhibitor of mTOR (PP242) blocked effects of FSK on cell size and on S6 phosphorylation as well as HIF1α induction (FIG. 4A, FIG. 9).

HIF has been shown to promote cell growth by stimulating glycolysis and by decreasing mitochondrial oxidative metabolism (Semenza, 2010). This metabolic shift-referred to as the Warburg effect—is thought to promote the accumulation of biomass associated with tumor growth while reducing reactive oxygen species that often accompany increases in metabolic activity (Vander Heiden et al., 2009). The inventors wondered whether triggering of the cAMP pathway promotes islet viability in part by reprogramming the metabolic activity of beta cells. Consistent with this idea, exposure of primary cultured islets to FSK increased lactate accumulation and secretion, measures of glycolysis and HIF activity in islets (Zehetner et al., 2008); as a result, FSK treatment also led to increases in intra-cellular ATP concentrations (FIG. 4B).

Having seen that HIF stimulates the expression of stress defense genes, the inventors tested whether activation of the cAMP pathway protects beta cells against oxidative stress. Treatment of INS-1 cells with hydrogen peroxide increased beta cell death by trypan blue exclusion and by measurement of cleaved caspase 3 amounts (FIG. 4C). Pre-incubation with FSK protected against cell death; these effects were reversed when mTORC1 activity was inhibited with rapamycin. In keeping with these results in INS-1 cells, exposure of cultured primary islets to FSK also increased HIF1α accumulation and HMOX1 expression in primary cultures of pancreatic islets; these effects were blocked by co-incubation with rapamycin (FIG. 4D).

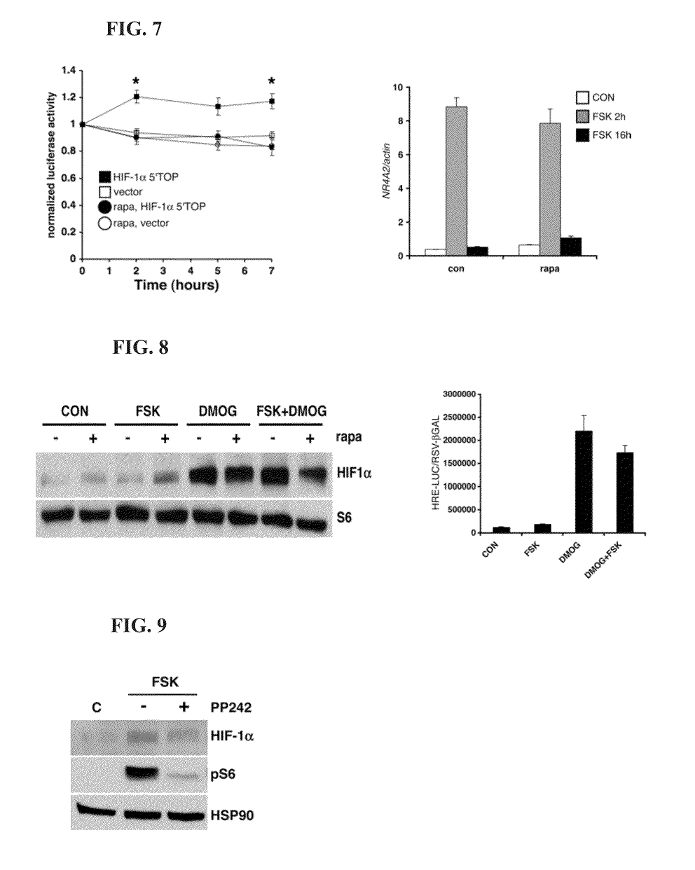

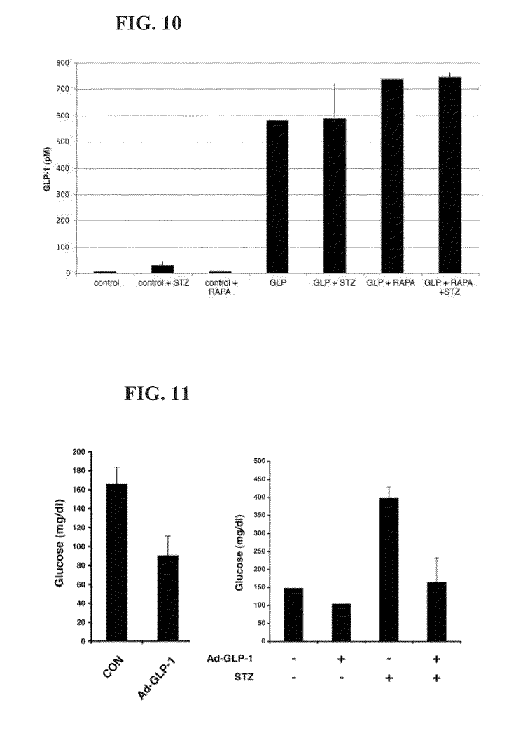

The inventors examined whether GLP-1 also regulates the mTOR pathway in vivo. Adenoviral expression of GLP-1 (Ad-GLP-1) in liver increased circulating levels of GLP-1, leading to corresponding decreases in circulating glucose concentrations relative to control (Ad-GFP) mice (FIG. 10, FIG. 11). Phospho-S6 staining—nearly undetectable in control mice—was substantially upregulated in pancreatic beta cells from mice expressing Ad-GLP-1 (FIG. 4E). The effects of Ad-GLP-1 on S6 phosphorylation appear mTOR-dependent because intra-peritoneal (IP) injection of rapamycin blocked GLP-1-dependent increases in phospho-S6 staining (FIG. 10).

Based on the ability for Ad-GLP-1 to stimulate the mTOR pathway in vivo, the inventors tested whether mTOR-HIF induction protects against the development of diabetes following administration of streptozotocin (STZ). Although absent from islets of control mice, beta cell apoptosis increased markedly in STZ-treated animals, leading to increases in circulating blood glucose concentrations (FIG. 4F, FIG. 11). Remarkably, Ad-GLP-1 expression blocked STZ-dependent increases in beta cell apoptosis and circulating glucose concentrations; these effects were reversed when rapamycin was co-administered (FIG. 4F, FIG. 11). Taken together, these experiments indicate that GLP-1 enhances islet function through activation of the mTOR-HIF pathway.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Altarejos, J. Y., and Montminy, M. (2011). CREB and the CRTC co-activators: sensors for hormonal and metabolic signals. Nat Rev Mol Cell Biol 12, 141-151.
Buse, J. B., Drucker, D. J., Taylor, K. L., Kim, T., Walsh, B., Hu, H., Wilhelm, K., Trautmann, M., Shen, L. Z., and Porter, L. E. (2010). DURATION-1: exenatide once weekly produces sustained glycemic control and weight loss over 52 weeks. Diabetes Care 33, 1255-1261.
Cantley, J., Selman, C., Shukla, D., Abramov, A. Y., Forstreuter, F., Esteban, M. A., Claret, M., Lingard, S. J., Clements, M., Harten, S. K., et al. (2009). Deletion of the von Hippel-Lindau gene in pancreatic beta cells impairs glucose homeostasis in mice. J Clin Invest 119, 125-135.
Cheng, K., Ho, K., Stokes, R., Scott, C., Lau, S. M., Hawthorne, W. J., O'Connell, P. J., Loudovaris, T., Kay, T. W., Kulkarni, R. N., et al. (2010). Hypoxia-inducible factor-1alpha regulates beta cell function in mouse and human islets. J Clin Invest 120, 2171-2183.
Choo, A. Y., Yoon, S. O., Kim, S. G., Roux, P. P., and Blenis, J. (2008). Rapamycin differentially inhibits S6Ks and 4E-BP1 to mediate cell-type-specific repression of mRNA translation. Proc Natl Acad Sci USA 105, 17414-17419.
Dentin, R., Hedrick, S., Xie, J., Yates, J., 3rd, and Montminy, M. (2008). Hepatic glucose sensing via the CREB coactivator CRTC2. Science 319, 1402-1405.
Drucker, D. J. (2006). The biology of incretin hormones. Cell Metab 3, 153-165.
Drucker, D. J., and Nauck, M. A. (2006). The incretin system: glucagon-like peptide-1 receptor agonists and dipeptidyl peptidase-4 inhibitors in type 2 diabetes. Lancet 368, 1696-1705.
Granot, Z., Swisa, A., Magenheim, J., Stolovich-Rain, M., Fujimoto, W., Manduchi, E., Mild, T., Lennerz, J. K., Stoeckert, C. J., Jr., Meyuhas, O., et al. (2009). LKB 1 regulates pancreatic beta cell size, polarity, and function. Cell Metab 10, 296-308.
Gunton, J. E., Kulkarni, R. N., Yim, S., Okada, T., Hawthorne, W. J., Tseng, Y. H., Roberson, R. S., Ricordi, C., O'Connell, P. J., Gonzalez, F. J., and Kahn, C. R. (2005). Loss of ARNT/HIF1beta mediates altered gene expression and pancreatic-islet dysfunction in human type 2 diabetes. Cell 122, 337-349.
Inada, A., Hamamoto, Y., Tsuura, Y., Miyazaki, J., Toyokuni, S., Ihara, Y., Nagai, K., Yamada, Y., Bonner-Weir, S., and Seino, Y. (2004). Overexpression of inducible cyclic AMP early repressor inhibits transactivation of genes and cell proliferation in pancreatic beta cells. Mol Cell Biol 24, 2831-2841.
Jhala, U.S., Canettieri, G., Screaton, R. A., Kulkarni, R. N., Krajewski, S., Reed, J., Walker, J., Lin, X., White, M., and Montminy, M. (2003). cAMP promotes pancreatic beta-cell survival via CREB-mediated induction of IRS2. Genes Dev 17, 1575-1580.
Koo, S. H., Flechner, L., Qi, L., Zhang, X., Screaton, R. A., Jeffries, S., Hedrick, S., Xu, W., Boussouar, F., Brindle, P., et al. (2005). The CREB coactivator TORC2 is a key regulator of fasting glucose metabolism. Nature 437, 1109-1111.
Mantovani, G., Bondioni, S., Ferrero, S., Gamba, B., Ferrante, E., Peverelli, E., Corbetta, S., Locatelli, M., Rampini, P., Beck-Peccoz, P., et al. (2005). Effect of cyclic adenosine 3′,5′-monophosphate/protein kinase a pathway on markers of cell proliferation in nonfunctioning pituitary adenomas. J Clin Endocrinol Metab 90, 6721-6724.
Norquay, L. D., D'Aquino, K. E., Opare-Addo, L. M., Kuznetsova, A., Haas, M., Bluestone, J. A., and White, M. F. (2009). Insulin receptor substrate-2 in beta-cells decreases diabetes in nonobese diabetic mice. Endocrinology 150, 4531-4540.
Park, S., Dong, X., Fisher, T. L., Dunn, S., Omer, A. K., Weir, G., and White, M. F. (2006). Exendin-4 uses Irs2 signaling to mediate pancreatic beta cell growth and function. J Biol Chem 281, 1159-1168.
Parsons, G. B., Souza, D. W., Wu, H., Yu, D., Wadsworth, S. G., Gregory, R. J., and Armentano, D. (2007). Ectopic expression of glucagon-like peptide 1 for gene therapy of type II diabetes. Gene Ther 14, 38-48.
Polak, P., and Hall, M. N. (2009). mTOR and the control of whole body metabolism. Curr Opin Cell Biol 21, 209-218.
Porat, S., Weinberg-Corem, N., Tornovsky-Babaey, S., Schyr-Ben-Haroush, R., Hija, A., Stolovich-Rain, M., Dadon, D., Granot, Z., Ben-Hur, V., White, P., et al. (2011). Control of pancreatic beta cell regeneration by glucose metabolism. Cell Metab 13, 440-449.
Ravnskjaer, K., Kester, H., Liu, Y., Zhang, X., Lee, D., Yates, J. R., 3rd, and Montminy, M. (2007). Cooperative interactions between CBP and TORC2 confer selectivity to CREB target gene expression. Embo J 26, 2880-2889.
Ringel, M. D., Schwindinger, W. F., and Levine, M. A. (1996). Clinical implications of genetic defects in G proteins. The molecular basis of McCune-Albright syndrome and Albright hereditary osteodystrophy. Medicine (Baltimore) 75, 171-184.
Ruvinsky, I., Sharon, N., Lerer, T., Cohen, H., Stolovich-Rain, M., Nir, T., Dor, Y., Zisman, P., and Meyuhas, O. (2005). Ribosomal protein S6 phosphorylation is a determinant of cell size and glucose homeostasis. Genes Dev 19, 2199-2211.
Screaton, R. A., Conkright, M. D., Katoh, Y., Best, J. L., Canettieri, G., Jeffries, S., Guzman, E., Niessen, S., Yates, J. R., 3rd, Takemori, H., et al. (2004). The CREB coactivator TORC2 functions as a calcium- and cAMP-sensitive coincidence detector. Cell 119, 61-74.
Semenza, G. L. (2010). HIF-1: upstream and downstream of cancer metabolism. Curr Opin Genet Dev 20, 51-56.
Sengupta, S., Peterson, T. R., and Sabatini, D. M. (2010). Regulation of the mTOR complex 1 pathway by nutrients, growth factors, and stress. Mol Cell 40, 310-322.
Vander Heiden, M. G., Cantley, L. C., and Thompson, C. B. (2009). Understanding the Warburg effect: the metabolic requirements of cell proliferation. Science 324, 1029-1033.
Zehetner, J., Danzer, C., Collins, S., Eckhardt, K., Gerber, P. A., Ballschmieter, P., Galvanovskis, J., Shimomura, K., Ashcroft, F. M., Thorens, B., et al. (2008). PVHL is a regulator of glucose metabolism and insulin secretion in pancreatic beta cells. Genes Dev 22, 3135-3146.
Zhang, X., Odom, D. T., Koo, S. H., Conkright, M. D., Canettieri, G., Best, J., Chen, H., Jenner, R., Herbolsheimer, E., Jacobsen, E., et al. (2005). Genome-wide analysis of cAMP-response element binding protein occupancy, phosphorylation, and target gene activation in human tissues. Proc Natl Acad Sci USA 102, 4459-4464.
Zoncu, R., Efeyan, A., and Sabatini, D. M. (2011). mTOR: from growth signal integration to cancer, diabetes and ageing. Nat Rev Mol Cell Biol 12, 21-35.

What is claimed is:

1. A method of treating diabetes in a subject comprising administering to the subject a GLP-1 agonist and an iron chelator in an amount effective to treat diabetes.

2. The method of claim 1, wherein the GLP-1 agonist is selected from the group consisting of exenatide, bydureon, liraglutide, albiglutide, taspoglutide, and lixisenatide.

3. The method of claim 2, wherein the GLP-1 agonist is exenatide.

4. The method of claim 1, wherein the iron chelator is selected from the group consisting of deferoxamine and deferasirox.

5. The method of claim 1, wherein the subject is a human.

6. The method of claim 1, wherein the subject has type II diabetes.

7. The method of claim 1, wherein the GLP-1 agonist is administered subcutaneously to the subject.

8. The method of claim 1, further comprising administering an additional diabetes therapy to the subject.

9. The method of claim 8, wherein the additional diabetes therapy comprises administration of insulin to the subject.

10. The method of claim 8, wherein additional diabetes therapy comprises administration of a dipeptidyl peptidase-4 inhibitor to the subject.

11. The method of claim 10, wherein the dipeptidyl peptidase-4 inhibitor is selected from the group consisting of metformin, sitagliptin (MK-0431), vildagliptin (LAF237), saxagliptin, linagliptin, dutogliptin, gemigliptin, berberine, and alogliptin.

12. A method of culturing beta islet cells in vitro, comprising contacting the beta islet cells with a GLP1 agonist and a HIF1 activator in an amount effective to promote survival of the beta cells.

13. The method of claim 12, wherein the GLP1 agonist is exenatide.

14. The method of claim 12, wherein the HIF1 activator is a cAMP agonist.

15. The method of claim 14, wherein the cAMP agonist is forskolin.

16. The method of claim 12, wherein the HIF1 activator is an iron chelator.

17. The method of claim 14, wherein the HIF1 activator is an iron chelator, and wherein the iron chelator is selected from the group consisting of deferoxamine and deferasirox.

18. The method of claim 12, wherein said amount is effective to promote proliferation of the beta islet cells.

19. The method of claim 12, further comprising administering a plurality of the cultured beta islet cells to a subject.

20. The method of claim 19, wherein the cultured beta islet cells are comprised in a pharmaceutical preparation.

21. The method of claim 20, wherein the pharmaceutical preparation is formulated for intravenous or subcutaneous administration.

22. The method of claim 19, wherein the subject is a human.

23. The method of claim 22, wherein the human has type II diabetes.

24. A pharmaceutical preparation comprising a GLP-1 agonist and an iron chelator.

25. The pharmaceutical preparation of claim 24, wherein the pharmaceutical preparation is formulated for intravenous or subcutaneous administration.

26. The pharmaceutical preparation of claim 24, wherein the GLP-1 agonist is selected from the group consisting of exenatide, bydureon, liraglutide, albiglutide, taspoglutide, and lixisenatide.

27. The pharmaceutical preparation of claim 26, wherein the GLP-1 agonist is exenatide.

28. The pharmaceutical preparation of claim 24, wherein the preparation comprises a cAMP agonist.

29. The pharmaceutical preparation of claim 28, wherein the cAMP agonist is forskolin.

30. The pharmaceutical preparation of claim 28, wherein the iron chelator is selected from the group consisting of deferoxamine and deferasirox.


SEQUENCE LISTING (39 sequences)

SEQ ID NO: 1 - protein, 39 aa, Artificial Sequence
  Synthetic Peptide
HGEGTFTSDLSKQMEEEAVRLFIEWLKNGGPSSGAPPPS

SEQ ID NO: 2 - protein, 30 aa, Artificial Sequence
  Synthetic Peptide
HXEGTFTSDVSSYLEGQAAKEFIAWLVKXX

SEQ ID NO: 3 - DNA, 22 bp, Artificial Sequence
  Synthetic Primer
gggcagtcaatggatgagagtg

SEQ ID NO: 4 - DNA, 20 bp, Artificial Sequence
  Synthetic Primer
gcgcagactttaggtgcatg

SEQ ID NO: 5 - DNA, 20 bp, Artificial Sequence
  Synthetic Primer
tgtttatgtggctcgcgctg

SEQ ID NO: 6 - DNA, 20 bp, Artificial Sequence
  Synthetic Primer
acaggcgtgctggctgacac

SEQ ID NO: 7 - DNA, 20 bp, Artificial Sequence
  Synthetic Primer
tgatgattcgggcaagtgcc

SEQ ID NO: 8 - DNA, 20 bp, Artificial Sequence
  Synthetic Primer
tggcaagaaggagagcggac

SEQ ID NO: 9 - DNA, 20 bp, Artificial Sequence
  Synthetic Primer
gtccacagaaggaacgtgtc

SEQ ID NO: 10 - DNA, 20 bp, Artificial Sequence
  Synthetic Primer
tctacaatgagctgcgtgtg

SEQ ID NO: 11 - DNA, 20 bp, Artificial Sequence
  Synthetic Primer
ggtctcaaacatgatctggg

SEQ ID NO: 12 - DNA, 20 bp, Artificial Sequence
  Synthetic Primer
gaaaaccaagcacatgctgc

SEQ ID NO: 13 - DNA, 20 bp, Artificial Sequence
  Synthetic Primer
ttgttgcacatcagcagcac

SEQ ID NO: 14 - DNA, 20 bp, Artificial Sequence
  Synthetic Primer
ctacctgtccaaactgttgg

SEQ ID NO: 15 - DNA, 20 bp, Artificial Sequence
  Synthetic Primer
ggtaaggtgtccaggaaaag

SEQ ID NO: 16 - DNA, 20 bp, Artificial Sequence
  Synthetic Primer
tctcccaaagtggcctacaa

SEQ ID NO: 17 - DNA, 20 bp, Artificial Sequence
  Synthetic Primer
tcatgggcatgtagccatca

SEQ ID NO: 18 - DNA, 20 bp, Artificial Sequence
  Synthetic Primer
aaggaagagctgagattcgc

SEQ ID NO: 19 - DNA, 20 bp, Artificial Sequence
  Synthetic Primer
ctcagacttggtgactgaca

SEQ ID NO: 20 - DNA, 20 bp, Artificial Sequence
  Synthetic Primer
gaagcagatctaccaacagc

SEQ ID NO: 21 - DNA, 20 bp, Artificial Sequence
  Synthetic Primer
cacagggtgactgaggtctt

SEQ ID NO: 22 - DNA, 20 bp, Artificial Sequence
  Synthetic Primer
accactgctaaggcatcagc

SEQ ID NO: 23 - DNA, 20 bp, Artificial Sequence
  Synthetic Primer
gctccttggatgagctttgt

SEQ ID NO: 24 - DNA, 21 bp, Artificial Sequence
  Synthetic Primer
gcttatgggtttctccaaact

SEQ ID NO: 25 - DNA, 20 bp, Artificial Sequence
  Synthetic Primer
gtgacacctcccccacatac

SEQ ID NO: 26 - DNA, 20 bp, Artificial Sequence
  Synthetic Primer
aggctttaagctggtgatgg

SEQ ID NO: 27 - DNA, 20 bp, Artificial Sequence
  Synthetic Primer
ataccagaaggccatgtcct

SEQ ID NO: 28 - DNA, 20 bp, Artificial Sequence
  Synthetic Primer
gaagaaggagaacctgaagg

SEQ ID NO: 29 - DNA, 20 bp, Artificial Sequence
  Synthetic Primer
acagagattcactggctgcg

SEQ ID NO: 30 - DNA, 20 bp, Artificial Sequence
  Synthetic Primer
aggaagtacacgagaagctc

SEQ ID NO: 31 - DNA, 20 bp, Artificial Sequence
  Synthetic Primer
ctccagtcacagaacctcca

SEQ ID NO: 32 - DNA, 20 bp, Artificial Sequence
  Synthetic Primer
gactgtggtactgagagcag

SEQ ID NO: 33 - DNA, 20 bp, Artificial Sequence
  Synthetic Primer
cctggcaaaggcttcccatt

SEQ ID NO: 34 - DNA, 20 bp, Artificial Sequence
  Synthetic Primer
ctgaggaagatcgacagact

SEQ ID NO: 35 - DNA, 20 bp, Artificial Sequence
  Synthetic Primer
gatatgggcaatccgtaacc

SEQ ID NO: 36 - DNA, 20 bp, Artificial Sequence
  Synthetic Primer
gtgcatcccatttccaccat

SEQ ID NO: 37 - DNA, 20 bp, Artificial Sequence
  Synthetic Primer
gagtcagtgtcaccttcaca

SEQ ID NO: 38 - DNA, 20 bp, Artificial Sequence
  Synthetic Primer
gcgcacagctactctcagca

SEQ ID NO: 39 - DNA, 21 bp, Artificial Sequence
  Synthetic Primer
gtcagacgccttccaatgtag